(19) United States
(12) Patent Application Publication
(10) Pub. No.: US 2009/0275773 A1
(43) Pub. Date: Nov. 5, 2009

(54) PROCESS FOR THE TRANSESTERIFICATION OF TRIGLYCERIDES

(75) Inventors: Frank Rossner, Oldenburg (DE); Oliver Meyer, Oldenburg (DE); Rainer Rakoczy, Rosenheim (DE); Richard Fischer, Bad Aibling (DE)

Correspondence Address: CONNOLLY BOVE LODGE & HUTZ LLP, 1875 EYE STREET, N.W., SUITE 1100, WASHINGTON, DC, 20006, US

(73) Assignee: Sud-Chemie AG, Munchen (DE)

(21) Appl. No.: 12/430,419

(22) Filed: Apr. 27, 2009

(30) Foreign Application Priority Data
Apr. 30, 2008 (DE) 10 2008 021 665.8

(51) Int. Cl. C07C 67/02

(52) U.S. Cl. 560/129

(57) ABSTRACT

The present invention relates to a process in which a triglyceride is transesterified with an alcohol in the presence of a solid catalyst. In order to provide a process by means of which a triglyceride can be transesterified with an alcohol in the presence of a solid catalyst in relatively moderate reaction conditions with a good yield and at relatively high reaction rate, it is proposed that the solid catalyst comprises a zeolite X with an Si/Al atomic ratio of less than 1.2.

The present invention relates to a process, in particular for the production of biodiesel, wherein in the process a triglyceride is transesterified with an alcohol in the presence of a solid catalyst.

The global demand for renewable fuels is very likely to clearly increase over the next few years. According to the Directive on the promotion of the use of biofuels (Directive 2003/30/EC of the European Parliament), by the end of 2010 alternative fuels must account for a minimum of 5.75% of fuels sold in the EU states. The demand for fuels which can be obtained from renewable raw materials will thus clearly increase. In addition to bioethanol, biodiesel is currently the only biofuel which is used to a significant extent.

Longer-chain fatty acid alkyl esters which are preferably produced by transesterification of triglycerides from natural sources with preferably methanol or ethanol are generally called biodiesels. The triglycerides are preferably used in the form of plant oils such as for example rape-seed oil or soya oil. During transesterification, batch processes and semi-continuous processes are used in particular. In addition, continuous processes for biodiesel production are also known in the state of the art, such as for example in U.S. Pat. No. 5,354,878 A and EP 562 504 A2. DE 196 22 601 C1 also describes a continuous process for biodiesel production employing used grease as starting material.

Within the framework of biodiesel production starting from triglycerides, homogeneous catalysts based on alkaline-metal hydroxides are almost exclusively used at present. A great disadvantage of these catalysts is that energy-intensive working-up and purification steps of the obtained product mixtures are required which account for the greatest part of the total energy requirement of the biodiesel production process.

In addition to homogeneously catalyzed processes for the production of biodiesel, heterogeneously catalyzed processes using solid catalysts are, however, also described in the state of the art. WO 2005/093015 A1 for example describes the transesterification of triglycerides using zinc aluminates of the spinell type of the general formula ZnAl₂O₄xZnOyAl₂O₃ (with x and y from 0 to 2) at reaction temperatures of 170 to 250° C. and a pressure of 30 to 60 bar.

Thus processes for the production of biodiesel by transesterification of triglycerides using solid contacts are known in the state of the art. These processes must however be carried out at relatively high temperatures and pressures in order to achieve economically workable reaction rates and yields.

The object of the present invention is therefore to provide a process by means of which a triglyceride can be transesterified with an alcohol in the presence of a solid catalyst under relatively moderate reaction conditions in a good yield at a relatively high reaction rate. This object is achieved starting from a process of the type according to the preamble in that the solid catalyst comprises a zeolite X with an Si/Al atomic ratio of less than 1.2.

Surprisingly it was found that triglycerides can be transesterified with alcohol at moderate temperatures and pressures in the presence of a solid catalyst which comprises a zeolite X with an Si/Al atomic ratio of less than 1.2, at a relatively high reaction rate.

Triglycerides are known in the state of the art. Within the framework of the present invention by “triglyceride” is meant as in the state of the art compounds of glycerol in which the three hydroxy groups are esterified with an acid. Within the framework of the present invention the acids are preferably carboxylic acids, for preference linear monocarboxylic acids, more preferably linear monocarboxylic acids with 4 to 26 carbon atoms and particularly preferably linear monocarboxylic acids with 12 to 22 carbon atoms.

By “alcohol” is meant within the framework of the present invention compounds which satisfy the general formula CₙH₂ₙ₊₁OH.

Solid catalysts are catalysts whose state of aggregation is the solid form. A distinction is drawn in the case of solid catalysts between complete catalysts which consist completely or almost completely of a catalytically active mass and supported catalysts in which the catalytically active mass is applied to a catalyst support. A further distinction is drawn in the case of solid catalysts depending on in which structural form these are present. The distinction is between solid catalysts formed as powder, shaped body or as monolith.

Solid catalysts in powder form preferably have an average particle size (d 50) of 1 μm to 100 μm and are mostly used in reactions which are carried out in non- or semi-continuously operating agitated-tank or fluid-bed reactors. Powdery solid catalysts can be both complete and supported catalysts.

Solid catalysts formed as shaped bodies, i.e. as three-dimensional bodies, which, just like powdery solid catalysts, can be present as complete or supported catalysts, are as a rule used in so-called fixed-bed reactors in which the educts can be continuously supplied and the resulting products removed.

Monolith catalysts, which can likewise be formed both as complete or as supported catalysts, generally have a honeycomb structure. Frequently they are developed as supported catalysts and comprise a honeycomb body which is coated with a so-called washcoat. While the honeycomb body mostly consists of a low surface area mineral ceramic, such as for example cordierite, or of metal, such as a metal sheet, the washcoat generally comprises a high surface area metal oxide or metal-oxide mixture which can be applied to the honeycomb body by means of a corresponding metal-oxide suspension. The washcoat is applied to the honeycomb body because the honeycomb body frequently has a relatively small surface, which is why without the washcoat only a relatively small amount of freely available catalytically active mass could be applied to the honeycomb body.

By “zeolite” is meant, according to the definition of the International Mineralogical Association (D. S. Coombs et al., Can. Mineralogist, 35, 1997, 1571) a crystalline substance from the group of the aluminium silicates with a spatial network structure of the general formula Mⁿ¹[(AlO₂)ₓ(SiO₂)_{y}]xH₂O, which is constructed from SiO_{4/2} and AlO_{4/2} tetrahedra, which are joined together by common oxygen atoms to form a regular three-dimensional network. The Si/Al atomic ratio is always greater than/equal to 1 according to the so-called “Löwenstein Rule”, which prevents the occurrence of two adjacent negatively-charged AlO_{4/2} tetrahedra.

The zeolite structure contains open cavities in the form of cages and channels which are characteristic of each zeolite type. The zeolites are divided into different structural types according to their topology. The cavities of the zeolite structure are normally occupied by water molecules and cations. An aluminium atom attracts an excess negative charge which is compensated for by these cations which can be exchanged. The inner surface of the zeolites represents the catalytically active surface. The more aluminium and the less silicon a zeolite contains, the denser is the negative charge in its lattice and the more polar its inner surface. The pore size and structure is determined, in addition to the parameters, during production (use or type of templates, pH, pressure, temperature, presence of seed crystals) by the Si/Al atomic ratio which determines the greatest part of the catalytic character of a zeolite.

Because of the presence of the trivalent aluminium cations as tetrahedron centre in the zeolite skeleton the zeolite receives a negative charge in the form of so-called anion spots in whose vicinity the corresponding cation positions are located. The negative charge is compensated for by incorporating cations into the pores of the zeolite material. Zeolites are mainly classified according to the geometry of the cavities which are formed by the rigid network of the SiO_{4/2}/AlO_{4/2} tetrahedra. The inlets to the cavities are formed from 8, 10 or 12 rings (narrow, average or wide-pored zeolites).

As already mentioned above, the cations can be exchanged in the cavities of zeolites. The aim of such a modification is frequently a desired change in the catalytic or adsorptive properties of the zeolite. The exchange behaviour of zeolites depends on many complex factors such as nature and size of the cations to be introduced or exchanged, temperature, concentration of the cations in the solution, the nature of the counteranions present in solution and structure of the zeolite in question. The maximum exchange capacity is determined by the Si/Al atomic ratio, wherein, generally, the smaller the Si/Al atomic ratio, the greater is the exchange capacity. The ion-sieve character of a zeolite can also influence the maximum ion-exchange capacity.

Zeolites of the faujasite (FAU) structural type have the so-called sodalite or β-cage as polyhedron structure. The sodalite units are connected to one another via hexagonal prisms, wherein a cage, also called supercage in the literature, is formed. The supercage can be accessed via a window formed by a 12-ring and has pore openings of 0.74 nm. Isotypic structures of faujasite are zeolite X with an Si/Al atomic ratio of 1 to 1.5 and zeolite Y with an Si/Al atomic ratio greater than 1.5 to 3.

The zeolite to be used within the framework of the present invention is a zeolite X with an Si/Al atomic ratio of less than 1.2, preferably a zeolite X with an Si/Al atomic ratio of less than 1.2 to 1.0. Such zeolites can for example be obtained commercially in alkaline form, in alkaline-earth form and in mixed alkaline/alkaline-earth form. Zeolite X with an Si/Al atomic ratio of less than 1.05, which in the literature are also called LSX (Low Silica X) zeolites, can for example be prepared according to the directions “GH Kühl, Zeolites 7 (1987) 451”. The Si/Al atomic ratio of the zeolite X to be used according to the invention can be determined by methods known to a person skilled in the art from the state of the art such as for example by complete decomposition of the zeolite followed by measurement of the decomposition using atomic absorption spectroscopy (AAS), atomic emission spectroscopy (AES) or inductively coupled plasma (ICP) or by means of ²⁹Si-MAS-NMR on the solid, wherein the latter (NMR) method is preferred according to the invention.

It was found that the smaller the Si/Al atomic ratio of the zeolite X, the greater is the activity of the solid catalyst to be used in the process according to the invention, with regard to the transesterification of triglycerides. According to a preferred embodiment of the process according to the invention the zeolite X therefore has an Si/Al atomic ratio of less than 1.15, preferably an Si/Al atomic ratio of less than 1.10, more preferably an Si/Al atomic ratio of less than 1.05 and yet more preferably an Si/Al atomic ratio of less than 1.02.

According to a further preferred embodiment of the process according to the invention the zeolite X has an Si/Al atomic ratio of 1.00.

According to a further preferred embodiment of the process according to the invention the alcohol is an alcohol with 1 to 8 carbon atoms. It was found that, with regard to the transesterification of triglycerides with alcohols with 1 to 8 carbon atoms, the zeolite X to be used in the process according to the invention is characterized by a particularly high activity.

In addition, it was found that by means of the process according to the invention, triglycerides can be transesterified with primary alcohols at a relatively high speed. According to a further preferred embodiment of the process according to the invention the alcohol is therefore a primary alcohol.

With regard in particular to the production of biodiesel the alcohol is selected from the group consisting of methanol, ethanol, propanol and butanol (butane-1-ol), wherein methanol and ethanol are particularly preferred. By means of the process according to the invention triglycerides can be transesterified in particular with the alcohols methanol, ethanol, propanol and butanol, preferably with methanol and ethanol, under particularly moderate reaction conditions such as temperature and pressure. According to a further preferred embodiment of the process according to the invention the alcohol is therefore selected from the group consisting of methanol, ethanol, propanol and butanol, preferably methanol and ethanol. If ethanol is used, thus it is preferred in particular on ecological grounds if the ethanol is bioethanol produced from renewable raw materials. Biodiesel produced from triglycerides and bioethanols is also called green biodiesel.

The transesterification of triglycerides with alcohol is a balanced reaction. In order to shift the balance of the reaction to the side of the fatty acid alkyl esters resulting from the transesterification and of the glycerol while preserving economically justifiable reaction conditions, according to a further preferred embodiment of the process according to the invention it is provided that the alcohol and the triglycerides are used in a molar ratio of 3:1 to 15:1, preferably in a molar ratio of 6:1 to 12:1.

According to a further preferred embodiment of the process according to the invention it is provided that the zeolite X is present in an alkaline form, in an alkaline-earth form or in a mixed alkaline/alkaline-earth form. It was shown that the zeolite X to be used in the process according to the invention shows a satisfactory activity with regard to the transesterification of triglycerides with alcohols in the alkaline form, and also in the alkaline-earth form and in the mixed alkaline/alkaline-earth form.

Alkaline form means that at least 60% of the cation positions of the zeolite X are occupied by alkaline-metal ions, preferably at least 70% of the cation positions, more preferably at least 90% of the cation positions and particularly preferably at least 95% of the cation positions.

Determining which cations occupy the cation positions of the zeolite and the extent to which the cation positions are occupied with the respective type of cation of the zeolite X to be used according to the invention can take place by means of methods known to the person skilled in the art from the state of the art such as for example by complete decomposition of the zeolite followed by measurement of the decomposition using AAS, AES or ICP, wherein the latter (ICP) method is preferred according to the invention.

Analogously to this alkaline-earth form means that at least 60% of the cation positions of the zeolite X, preferably at least 70% of the cation positions, more preferably at least 90% of the cation positions and particularly preferably at least 95% of the cation positions are occupied by alkaline-earth metal ions.

Correspondingly, a mixed alkaline/alkaline-earth form of the zeolite X means that at least 60% of the cation positions of the zeolite X are occupied by alkaline-metal and alkaline-earth metal ions. Preferably, at least 70% of the cation positions of the zeolite X are occupied by alkaline-metal and alkaline-earth metal ions, more preferably at least 80% of the cation positions, even more preferably at least 90% of the cation positions and particularly preferably at least 95% of the cation positions.

According to a further preferred embodiment of the process according to the invention the zeolite X is present in the alkaline form and cation positions of the zeolite X are occupied by Li, Na, K, Rb and/or Cs ions. It was shown that, in the transesterification of triglycerides, the zeolite X shows a good activity within the framework of the process according to the invention in the alkaline form. The cation positions of the zeolite X can be occupied by Li, Na, K, Rb or Cs ions and also with two or more different alkaline-metal ions of those named above. For example the cation positions of the zeolite can be occupied by Na and K ions or with K and Rb and/or Cs ions.

According to a further preferred embodiment of the process according to the invention, cation positions of the zeolite X are occupied by K ions. It was found that, with regard to the transesterification of triglycerides with alcohols, the zeolite X to be used in the process according to the invention is characterized by a particularly high activity in the K form.

In addition it was found that, with regard to the transesterification of triglycerides, the more cation positions are occupied by K ions, the greater is the activity of the zeolite X to be used in the process according to the invention. According to a further preferred embodiment of the process according to the invention, at least 90% of the cation positions of the zeolite X are occupied by K ions, preferably at least 95% of the cation positions and particularly preferably at least 99% of the cation positions.

In addition it was shown that, with regard to the transesterification of triglycerides, the activity of zeolites X with an Si/Al atomic ratio of less than 1.2 can be significantly increased if at least 2% of the cation positions of the zeolite X are occupied by Rb and/or Cs ions. According to a further preferred embodiment of the process according to the invention it is provided that at least 2% of the cation positions of the zeolite X are occupied by Rb and/or Cs ions. It is particularly preferred in this context if the greatest part of the cation positions is occupied by Na and/or K ions and 2 to 10% of the cation positions of the zeolite X with Rb ions, Cs ions or Rb and Cs ions.

If it is provided, in the process according to the invention, to use the zeolite X in the alkaline-earth form, then cation positions of the zeolite X are occupied by Be, Mg, Ca, Sr and/or Ba ions. The cation positions can be occupied by Be, Mg, Ca, Sr or Ba ion and also with two or more of the above-named alkaline-earth metal ions. In this context it is particularly preferred that the cation positions of the zeolite X are occupied by Ca ions, i.e. that the zeolite X is present in the Ca form.

If it is provided to use the zeolite X to be used in the process according to the invention in the mixed alkaline/alkaline-earth form, then cation positions of the zeolite X are occupied by at least one alkaline-metal ion selected from the group consisting of Li, Na, K, Rb and Cs ions and by at least one alkaline-earth metal ion selected from the group consisting of Be, Mg, Ca, Sr and Ba ions. Examples of preferred mixed alkaline/alkaline-earth forms are preferably the occupation of the cation positions of the zeolite X by K and Mg ions; K and Ca ions; K, Na and Mg ions; K, Na and Ca ions.

According to a further preferred embodiment of the process according to the invention it is provided that the transesterification is carried out at a temperature of 25° C. to 150° C., preferably at a temperature of 65° C. to 100° C. It was found that at a temperature below 25° C. the transesterification proceeds at only a relatively low speed, while at a temperature above 150° C. no significant increase in the reaction rate can be achieved.

According to a further preferred embodiment of the process according to the invention it is provided that the transesterification is carried out at the boiling point of the alcohol. By boiling point of the alcohol is thus meant the boiling point of the alcohol under the respective conditions (such as e.g. the pressure). This measure is simple to achieve in process engineering terms and thus particularly favourable in cost terms, wherein simultaneously a relatively high reaction rate is achieved in the transesterification of the triglycerides.

It is simple to achieve in terms of process engineering and thus favourable in cost terms if the process according to the invention is carried out at ambient pressure. Correspondingly it is provided according to a further preferred embodiment of the process according to the invention that the process is carried out at ambient pressure, which generally corresponds to normal pressure.

Alternatively it can be provided that the process is carried out at a slight above-ambient pressure of up to 2 bar, for example at a pressure of 1.1 to 2 bar, in order to increase the rate of the transesterification reaction.

The process according to the invention can be carried out both continuously and also discontinuously. If the process is to be carried out continuously, it may be preferred to introduce the solid catalyst first in the form of a fixed bed. In this connection it can be provided according to a further preferred embodiment of the process according to the invention that the solid catalyst is formed as a shaped body. All customary shaped bodies may come into consideration as shaped bodies, preferably cylinders, hollow cylinders, spheres, rings, stars, tablets, cartwheels, inverted cartwheels, trilobes, tetralobes etc.

It was found that the solid catalyst to be used in the process according to the invention shows a clear activity in the transesterification of triglycerides with a percentage by weight of only 0.5 wt.-% of zeolite X, wherein the greater the proportion of corresponding zeolite X the higher is the activity of the solid catalyst. According to the invention it is preferred that the solid catalyst comprises 0.5 to 100 wt.-% of the zeolite X, more preferably 50 to 98 wt.-%, yet more preferably 70 to 95 wt.-% and particularly preferably 80 to 90 wt.-%.

According to a further preferred embodiment of the process according to the invention it is provided that the solid catalyst comprises CaO and/or MgO. It was shown that CaO, MgO as well as CaO and MgO can further improve the activity of the solid catalyst with regard to the transesterification of triglycerides.

The proportion of CaO, MgO or CaO and MgO in the solid catalyst is preferably 0.5 to 10 wt.-%, preferably 1 to 8 wt.-% and particularly preferably 2 to 6 wt.-%.

For the most cost-favourable production possible of for example biodiesel it is provided according to a further preferred embodiment of the process according to the invention that the triglyceride is of plant or animal origin, wherein both oils and greases can come into consideration.

According to a further preferred embodiment of the process according to the invention the triglyceride is used in the process in the form of a plant or animal oil. Preferred examples of plant oils are palm oil, rape-seed oil and soya oil, wherein rape-seed oil is particularly preferred with regard to a particularly cost-favourable production of biodiesel.

According to a further preferred embodiment of the process according to the invention the proportion of the zeolite X in the reaction mixture located for example in a continuously or discontinuously operated reactor is 5 to 20 wt.-% relative to the weight of the triglyceride used. A largely complete reaction of the triglyceride in a relatively short time is thereby guaranteed.

In order to produce for example large quantities of biodiesel by means of the process according to the invention it is provided according to a further preferred embodiment of the process according to the invention that the process is carried out in continuous operation.

Corresponding to a particularly preferred embodiment a triglyceride, preferably in the form of a plant oil, preferably rape-seed oil, is transesterified with methanol in the presence of a solid catalyst in the process according to the invention, wherein the solid catalyst comprises a zeolite X with an Si/Al atomic ratio of less than 1.05, wherein at least 90% of the cation positions are occupied by K and/or Na ions, preferably by K ions, wherein the process is preferably carried out at a temperature above 65° C.

The examples below serve, in conjunction with the drawing, to describe the invention. There is shown in:

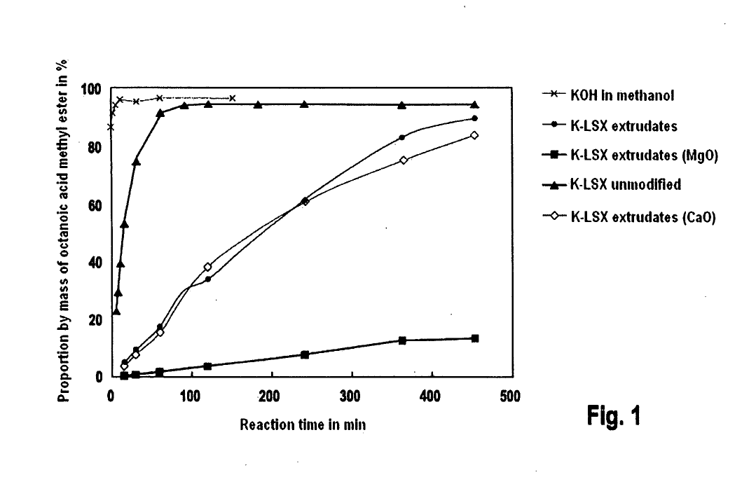

FIG. 1: a graphic representation of the proportions by mass in percent of octanoic acid methyl ester obtained during the transesterification of glycerol trioctanoate depending on the reaction time with regard to the homogeneous catalyst KOH (curve identified by crosses) and the solid catalysts “K-LSX unmodified” (curve identified by triangles), K-LSX extrudates (curve identified by circles), K-LSX extrudates (CaO) (curve identified by diamonds), K-LSX extrudates (MgO) (curve identified by squares).

### EXAMPLE 1

40 g of a pseudoboehmite called Pural SB, 150 g completely deionized water, 50 g 52% nitric acid, 300 g of a freshly ground zeolite X with an Si/Al atomic ratio of 1.02, whose cation positions were 93% occupied by K ions and 3.5% occupied by Na ions (hereinafter also called “K-LSX unmodified”), and 19 g of an extruding oil called “ExxOL oil” were processed by means of a kneader to form a plastic mass.

Extrudates were produced from this mass by means of an extruder with a 1/16-inch punched disk. 2-mm long cylinders were made from the extrudates.

The cylindrical shaped bodies obtained as described above were dried over a period of 12 h at 120° C. and calcined over a period of five hours at a temperature of 480° C. to activate the solid catalyst, wherein the temperature of 480° C. was set with a heat-up rate of 1° C. per minute. The thus-obtained solid catalyst is called K-LSX extrudate.

### EXAMPLE 2

A solid catalyst was prepared according to example 1, wherein 25.4 g calcium acetate hydrate was added as an additional component to the extruded mass. The calcium acetate hydrate was dissolved in the water to be added to the mixture before the addition of the water to the mass.

The calcium acetate used was converted into CaO during calcination. This solid catalyst is called K-LSX extrudate (CaO).

### EXAMPLE 3

A solid catalyst was prepared according to example 1, wherein 32.1 g magnesium acetate tetrahydrate, which was dissolved in the water to be added to the mass, was added as an additional component to the extruded mass.

The magnesium acetate was converted into MgO during calcination. This solid catalyst is called K-LSX extrudate (MgO).

### EXAMPLE 4

To investigate the catalytic activity of the solid catalysts “K-LSX unmodified”, K-LSX extrudate, K-LSX extrudate (CaO) and K-LSX extrudate (MgO) in the transesterification of triglycerides, the transesterification of triglyceride trioctanoate—as a defined triglyceride—with methanol was selected as test reaction. The reaction was carried out at normal pressure and at a temperature of 90° C. with a methanol-to-triglyceride molar ratio of 9:1 and with a weight ratio of zeolite X to triglyceride of 1:10. The results of the transesterification reactions are represented graphically in FIG. 1. The courses of the curves with regard to the solid catalysts K-LSX extrudate (line identified by circles) and K-LSX extrudate (CaO) (line identified by non-filled-in diamonds) show a similar course and prove the very high activity of these catalysts in the transesterification reaction of triglycerides. The course of the curve with regard to the catalyst “K-LSX unmodified” (line identified by triangles) shows a higher activity compared with the previously discussed catalysts, which is presumably attributable to the greater surface area of the zeolite powder compared with the extrudate and a concomitant reduced diffusion limitation. The course of the curve with regard to the catalyst K-LSX extrudate (MgO) (line identified by squares) shows a clearly reduced activity compared with the CaO-containing catalyst, which could be attributed to the relatively high MgO content of the catalyst.

### COMPARISON EXAMPLE

The transesterification of triglyceride trioctanoate with methanol in the presence of KOH was chosen as comparison example. The reaction was carried out at normal pressure and at a temperature of 70° C. with a methanol-to-triglyceride molar ratio of 6:1 and with a KOH content of 1.3 wt.-% relative to the weight of the methanol used and triglyceride trioctanoate. The result of the transesterification reaction is likewise represented in FIG. 1. The course of the curve (line identified by crosses) shows the known high activity of the homogeneous catalyst KOH in the transesterification reaction of triglycerides.

1. A process in which a triglyceride is transesterified with an alcohol in the presence of a solid catalyst, characterized in that the solid catalyst comprises a zeolite X with an Si/Al atomic ratio of less than 1.2.

2. The process according to claim 1, characterized in that the zeolite X has an Si/Al atomic ratio of less than 1.15.

3. The process according to claim 1, characterized in that the zeolite X has an Si/Al atomic ratio of 1.00.

4. The process according to claim 1, characterized in that the alcohol is an alcohol with 1 to 8 carbon atoms.

5. The process according to claim 1, characterized in that the alcohol is a primary alcohol.

6. The process according to claim 1, characterized in that the alcohol is selected from the group consisting of methanol, ethanol, propanol and butanol.

7. The process according to claim 1, characterized in that the alcohol is methanol or ethanol.

8. The process according to claim 1, characterized in that the alcohol and the triglyceride are used in a molar ratio of 3:1 to 15:1.

9. The process according to claim 1, characterized in that the zeolite X is present in an alkaline form, in an alkaline-earth form or in a mixed alkaline/alkaline-earth form.

10. The process according to claim 9, characterized in that the zeolite X is present in the alkaline form and cation positions of the zeolite X are occupied by Li, Na, K, Rb and/or Cs ions.

11. The process according to claim 9, characterized in that cation positions of the zeolite X are occupied by K ions.

12. The process according to claim 9, characterized in that at least 90% of the cation positions of the zeolite X are occupied by K ions.

13. The process according to claim 9, characterized in that at least 2% of the cation positions of the zeolite X are occupied by Rb and/or Cs ions.

14. The process according to claim 9, characterized in that the zeolite X is present in the alkaline-earth form and cation positions of the zeolite X are occupied by Be, Mg, Ca, Sr and/or Ba ions.

15. The process according to claim 14, characterized in that the cation positions of the zeolite X are occupied by Ca ions.

16. The process according to claim 9, characterized in that the zeolite X is present in the mixed alkaline/alkaline-earth form and cation positions of the zeolite X are occupied by at least one alkaline-metal ion selected from the group consisting of Li, Na, K, Rb and Cs ions and by at least one alkaline-earth metal ion selected from the group consisting of Be, Mg, Ca, Sr and Ba ions.

17. The process according to claim 1, characterized in that the transesterification is carried out at a temperature of 25° C. to 150° C.

18. The process according to claim 1, characterized in that the transesterification is carried out at the boiling point of the alcohol.

19. The process according to claim 1, characterized in that the transesterification is carried out at ambient pressure or at an above-ambient pressure of 1.1 to 2.0 bar.

20. The process according to claim 1, characterized in that the solid catalyst is formed as a shaped body.

21. The process according to claim 1, characterized in that the proportion of zeolite X in the solid catalyst is at least 70 wt.-%.

22. The process according to claim 1, characterized in that the solid catalyst contains CaO and/or MgO.

23. The process according to claim 22, characterized in that the proportion of CaO, MgO or CaO and MgO in the solid catalyst is 0.5 to 10 wt.-%.

24. The process according to claim 1, characterized in that the triglyceride is of plant or animal origin.

25. The process according to claim 1, characterized in that the triglyceride is used in the form of a plant or animal oil.

26. The process according to claim 1, characterized in that the proportion of zeolite X is 5 to 20 wt.-% relative to the weight of the triglyceride.

27. The process according to claim 1, characterized in that the process is carried out in continuous operation.
